(19) United States
(12) Patent Application Publication
(10) Pub. No.: US 2011/0217380 A1
(43) Pub. Date: Sep. 8, 2011

(54) PROTEINS THAT STIMULATE THE SECRETION OF SATIETY HORMONES

(75) Inventors: Maria Christiana Peter Geraedts, Swalmen (NL); Frederik Jan Troost, Maastricht (NL); Wilhelmus Hermanus Maria Saris, Meerssen (NL)

(73) Assignee: Universiteit Maastricht, Maastricht (NL)

(21) Appl. No.: 12/739,827

(22) PCT Filed: Oct. 26, 2008
(86) PCT No.: PCT/EP08/64493

§ 371 Date: Sep. 30, 2010

(30) Foreign Application Priority Data
Oct. 26, 2007 (EP) 07119403.9
Oct. 7, 2008 (EP) 08166045.8

(51) Int. Cl. A61K 38/02; A61K 9/00; A61K 9/14; A61P 3/04

(52) U.S. Cl. 424/489; 514/1.1; 424/400; 514/4.9

(57) ABSTRACT

The invention is in the field of weight management, in particular in the field of weight management by influencing the mechanisms of body-weight regulation. Intact pea protein and intact wheat protein were found to be effective in reducing appetite or inducing or increasing satiety when brought into contact with their receptors in the duodenum. Since it is known that intact proteins hydrolyse in the gastrointestinal tract, intact pea protein and intact wheat protein will not exhibit their satiating effect when ingested in a conventional oral preparation. Therefore, special care should be taken to deliver the intact proteins to the duodenum in order for them to arrive there intact. One object of the invention may therefore be achieved by incorporating the intact protein in an enteric delivery vehicle.

### FIELD OF THE INVENTION

The invention is in the field of weight management, in particular in the field of weight management by influencing the mechanisms of body-weight regulation. In particular, it relates to the use of protein compositions for inducing or increasing satiety in an animal or a human being.

### BACKGROUND OF THE INVENTION

Obesity is one of the major biomedical problems of the last few decades. It is important to find a treatment that affects the mechanisms of body-weight regulation.

The role of proteins in the regulation of long-term energy balance and maintenance of healthy body weight in humans has received little attention. There is some evidence that proteins play an important role in the regulation of food intake and body weight maintenance.

Food ingestion triggers a number of stimuli within the gastrointestinal tract that modulate appetite-sensations, such as the release of the gastrointestinal hormones cholecystokinin (CCK) and glucagon-like peptide 1 (GLP-1). CCK is produced by I-cells in the duodenal and jejunal mucosa, and secreted in response to luminal nutrients, especially lipids and proteins (1). GLP-1 is produced primarily by the L-cells in the distal small intestine and colon. Ingested nutrients stimulate CCK- and GLP-1 secretion by indirect, duodenally activated neurohumoral mechanisms, as well as by direct contact within the distal intestine (2).

The macronutrient composition of the diet plays an important role in the release of satiety hormones. Recent literature describes the positive role of dietary protein in reducing food intake by improving satiety sensations (3, 4). It seems that protein has the highest satiating effect when compared to other macronutrients in humans and rats (5, 6), although the nature of the protein can influence the satiating effects. However, it is unknown which proteins affect secretion of CCK and GLP-1.

Ingestion of a high-protein diet induces a significant rise in circulating levels of the gut hormones CCK and GLP-1 in the blood. This indicates that proteins play an important role in the secretion of these hormones. Several studies show that ingestion of high protein meals, satiety and fullness were higher, whereas hunger, appetite, desire to eat, and estimated quantity to eat were lower than the normal protein diet (11, 12).

The effects of several proteins on food intake and subjective ratings of hunger and fullness and on gastrointestinal hormone responses like glucagon-like peptide 1 (GLP-1) were investigated previously. Energy intake from a buffet meal ad libitum was significantly less after a whey-containing preload, and plasma levels of GLP-1 increased by 60-65% following the whey preload compared with an equivalent casein preload. The whey test meal induced an increase in satiety, which implicates that GLP-1 serves as a mediator of the increased satiety response to whey.

GLP-1 acts on stimulation of glucose-dependent insulin secretion and insulin biosynthesis, inhibition of glucagon secretion and gastric emptying, and inhibition of food intake (5). GLP-1 is released in response to nutrient ingestion from endocrine cells distributed throughout the small and large intestine. Following an initial nutrient-stimulated rise in circulating levels of GLP-1, the levels fall rapidly, largely due to renal clearance and the N-terminal degradation of the peptide by dipeptidyl peptidase IV (DPP IV/CD26; EC 3.4.14.5).

DPP IV is a 110 kDa plasma membrane glycoprotein ectopeptidase that belongs to the serine protease family. In mammals, DPP IV is ubiquitously expressed on the surface of endothelial and epithelial cells and highest levels in humans have been reported to occur in the intestine, bone marrow, and kidney. The enzymatic action of DPP IV is important for the brake down of protein hormones and has the capacity to inactivate or modulate gastric inhibitory peptide, glucagon-like peptide (GLP) 1 and 2, and neuropeptide Y, among others. DPP IV in the intestinal brush-border cleaves GLP-1 to an inactive form, and emerging evidence has supported the hypothesis that DPP IV is implicated in the regulation of glucose serum levels ad the control of appetite and satiety.

### SUMMARY OF THE INVENTION

This study demonstrates that intact dietary proteins can directly influence release of the satiety hormones CCK and GLP-1 in human duodenal tissue. We demonstrate herein that intact pea protein and intact wheat protein elevate both CCK and GLP-1 release. Both CCK and GLP-1 release was found to be elevated after exposing duodenal tissue to intact pea protein and intact wheat protein. This was shown in an ex vivo study using human duodenal tissue in an Ussing chamber. These results are confirmed in another in vitro study using a semi high throughput system for gut hormone secretion. This satiating effect was not observed when pea protein hydrolysates or wheat protein hydrolysates were used.

This leads us to conclude that intact pea protein and intact wheat protein are suitable for reducing appetite and/or induce or increase satiety when brought into contact with their receptors in the duodenum. Since it is known that intact proteins hydrolyse in the gastrointestinal tract, in particular in the stomach, intact pea protein and intact wheat protein will exhibit their satiating effect in the intestines only very partially if at all, when ingested in a conventional oral preparation. Therefore, special care should be taken to deliver the intact proteins to the duodenum in order for them to arrive there intact. Therefore, the intact pea protein and intact wheat proteins may be provided with an enteric coating.

The invention therefore relates to intact pea protein or intact wheat protein for use in the treatment and/or prophylaxis of obesity wherein said intact wheat protein or intact pea protein is provided with an enteric coating.

An enteric coating is a barrier applied to oral medication or oral foodstuff that controls the location in the digestive system where it is absorbed. Enteric refers to the small intestine; therefore enteric coatings prevent release of medication of foodstuff before it reaches the small intestine. Most enteric coatings work by presenting a surface that is stable at the highly acidic pH found in the stomach, but breaks down rapidly at the less acidic (relatively more basic) pH of the small intestines.

Although enterically coated proteins are a very suitable way of delivering proteins to the intestines, other delivery vehicles capable of protecting the proteins against hydrolysis in the stomach may also be suitable, depending on the particular effects envisaged. The skilled person will be aware of the most suitable delivery vehicle for a particular purpose.

Therefore, the invention also relates to intact pea protein or intact wheat protein for use in the treatment and/or prophylaxis of obesity wherein said intact wheat protein or intact pea protein is incorporated in a delivery vehicle that increases the resistance of said intact proteins against hydrolysis.

In particular, the intact proteins may be contained in particles in order to be effectively delivered at the duodenum.

The invention therefore relates to the use of intact wheat protein or intact pea protein for inducing or increasing satiety in a human or in an animal, wherein said intact wheat protein or intact pea protein is incorporated in particles that increase the resistance of said intact proteins against hydrolysis.

The invention also relates to the use of a food composition comprising intact wheat protein or intact pea protein for inducing satiety in a human being or in an animal wherein said intact wheat protein or intact pea protein is incorporated in a delivery vehicle such as a particle that increases the resistance of said intact proteins against hydrolysis.

The invention also relates to intact wheat protein or intact pea protein for use in the treatment and/or prophylaxis of obesity wherein said intact wheat protein or intact pea protein is incorporated in particles that increase the resistance of said intact proteins against hydrolysis.

The invention also relates to particles comprising between 1% and 100% intact wheat protein or intact pea protein as a fraction of their total protein content.

The invention also relates to a food composition comprising such particles.

### DETAILED DESCRIPTION OF THE INVENTION

A semi high throughput system for gut hormone secretion was used to study the role of dietary proteins in hormone secretion. Proteins were selected from the group consisting of ovomucoid, soybean, egg, wheat protein hydrolysate, whey, intact pea protein, casein hydrolysate, sodium caseinate, cod fish, and intact wheat protein.

The effect of the dietary proteins on CCK and GLP-1 secretion was investigated by exposing them to STC-1 cells, a murine intestinal cell line. STC-1 cells were incubated with a 1% protein solution in HBSS buffer for 2 hours. The supernatant was collected, and CCK and GLP-1 concentrations were determined using RIA assays. All analyses were performed in duplicate.

Effects of the different dietary proteins on GLP-1 and CCK release were compared with the negative control situation (only HBSS buffer). Intact wheat protein and intact pea protein induced an increase in GLP-1 release (5306.24 pM and 1191.24 pM respectively) compared to negative control (123.6 pM). CCK levels were increased after addition of intact wheat protein (51.5 pM) compared to negative control (20.5 pM).

Intact wheat protein induced a large increase in CCK and GLP-1 secretion, whereas intact pea protein resulted in increased GLP-1 levels. Therefore we conclude that intact wheat protein and intact pea protein may be used in the treatment of obesity and/or for inducing or increasing satiety.

Serine proteases such as trypsin and dipeptidyl peptidase IV (DPP IV) have been shown to be involved in the regulation of the release and activation of CCK and GLP-1. We found that intact pea protein is able to inhibit the activity of serine proteases, and thereby stimulate the secretion of CCK and GLP-1 (FIG. 1).

Six proteins (egg protein, egg protein hydrolysate, ovomucoid, intact pea protein, soybean, and whey protein) were tested for their inhibitory effects on the activity of DPP IV. Ovomucoid, intact pea protein, egg protein, egg protein hydrolysate, and whey protein inhibited DPP IV activity, with a remaining activity of 83.5%±1.5, 9.6%±0.5, and 67.0%±4.8 respectively (FIG. 1). Intact pea protein performed best in this assay.

Eleven naturally occurring dietary proteins (casein hydrolysate, codfish, egg, egg hydrolysate, ovomucoid, pea, sodium casein, soybean, wheat, wheat hydrolysate, and whey) were tested on their effects on satiety hormone secretion by incubating STC-1 cells with either protein and human trypsin or DPP IV. CCK and GLP-1 levels were determined in the supernatant.

We conclude that intact wheat protein or intact pea protein may be used in the treatment of overweight and obesity, due to their strong positive effects on satiety hormone release.

The present study is the first to show that specific intact proteins are able to influence satiety. DPP IV activity is, however, not a direct marker for the effects of the proteins on GLP-1 levels. Intact pea protein was found to be a strong inhibitor of human DPP IV, but the protein itself showed no effect on GLP-1 release compared to the negative control after 2 hours. Addition of the protein in combination with DPP IV resulted in a decrease of GLP-1 release. Another remarkable finding is that soybean, a well-known serine protease inhibitor, is not able to inhibit DPP IV activity. Another protein, egg protein hydrolysate, is able to inhibit DPP IV activity, and is able to stimulate GLP-1 release in STC-1 cells, but the combination of egg protein and DPP IV to the cells showed a decrease in GLP-1 levels. This might be due to the inhibitory capacity of the protein. Egg protein hydrolysate and intact pea protein might inhibit DPP IV in a competitive manner, but the enzyme favours GLP-1 prior to the protein.

The inhibition of DPP IV may result in beneficial effects on health, in particular in obese patients. However, so far only chemically synthesised inhibitors are known, and these are not commercially available. McIntosh et al. (McIntosh C H, Demuth H U, Pospisilik J A, Pederson R. Dipeptidyl peptidase IV inhibitors: how do they work as new antidiabetic agents? Regul Pept 2005; 128: 159-165.) found that oral treatment of DPP IV inhibitor over a 12-week period in DM2 rats had no effect on water or nutrient ingestion, but body weight was decreased by 12.5%. Inhibitor treated diabetic animals showed a marked improvement in glucose tolerance and increased insulin secretion.

DPP IV may be inhibited in several different ways. Competitive, non-competitive, mixed-type, and irreversible inhibition can occur (Lorey S, Stockel-Maschek A, Faust J, Brandt W, Stiebitz B, Gorrell M D, et al. Different modes of dipeptidyl peptidase IV (CD26) inhibition by oligopeptides derived from the N-terminus of HIV-1 Tat indicate at least two inhibitor binding sites. Eur J Biochem 2003; 270: 2147-2156). In most DPP IV inhibition studies, chemically synthesized compounds are used, which inhibit DPP IV either irreversible or in a mixed-type (Bauvois B. A collagen-binding glycoprotein on the surface of mouse fibroblasts is identified as dipeptidyl peptidase IV. Biochem J 1988; 252: 723-731, Farriol M, Pita A M, Fernandez-Bustos M A, Delgado G. Dipeptidyl-peptidase IV in patients with short bowel syndrome. Clin Nutr 2005; 24: 1099-1104, Lugari R, Dei Cas A, Ugolotti D, Barilli A L, Camellini C, Ganzerla G C, et al. Glucagon-like peptide 1 (GLP-1) secretion and plasma dipeptidyl peptidase IV (DPP-IV) activity in morbidly obese patients undergoing biliopancreatic diversion. Horm Metab Res 2004; 36: 111-115). The present study is the first to show that naturally occurring dietary proteins are able to inhibit the activity of human DPP IV.

Intact pea protein significantly inhibited DPP IV enzyme activity. Only less than 10% of its activity remained. Other proteins that were able to significantly inhibit the enzyme activity were ovomucoid, whey protein, egg protein, and egg protein hydrolysate. Soybean did not show a decrease in the activity of DPP IV.

Levels of the bioactive form of the satiety hormone GLP-1 present in the blood circulation fall rapidly, due to the degradation of the peptide by DPP IV. GLP-1 plays an important role in the ileal brake, resulting in satiety and weight reduction. Previous studies suggest that GLP-1 secretion is reduced in obese subjects and that weight loss normalizes the levels. The anorectic effects of GLP-1 are, however, preserved in obesity (Stanley S, Wynne K, Bloom S. Gastrointestinal satiety signals III. Glucagon-like peptide 1, oxyntomodulin, peptide YY, and pancreatic polypeptide. Am J Physiol Gastrointest Liver Physiol 2004; 286: G693-697.).

Addition of codfish protein, egg protein, egg protein hydrolysate, sodium casein, and intact wheat protein increased GLP-1 release in STC-1 cells, but in combination with DPP IV, egg protein hydrolysate was not to inhibit the enzyme activity, and the release of GLP-1 decreased significantly. There where no increased levels of GLP-1 observed after addition of the combination of proteins with DPP IV. This is due to the use of a “total GLP-1 RIA”. It is not known whether the cells are able to activate the secreted GLP-1, therefore the total GLP-1 RIA was chosen.

We found that adding codfish, egg protein, sodium casein, intact wheat protein, or a combination of these proteins to the diet induced a decrease of DPP IV activity. This will delay GLP-1 degradation, which causes GLP-1 levels to remain elevated during and after a meal containing any of these proteins and result in increased satiety sensations during and after a meal. The increased satiety sensations will decrease the amount of food intake, an on the long term, weight reduction may be induced. Inhibition of DPP IV by dietary proteins may also improve the glucose tolerance and insulin secretion in diabetes mellitus type 2 patients.

A further ex vivo study focused on the effects of proteins on hormone release from human intestinal tissue, using Ussing chamber technology (7-9). An important difference between our study and the in vivo studies performed in the prior art is that we tested intact proteins directly on duodenal tissue. In the in vivo studies described in the prior art, oral administration of the proteins occurs. Intact proteins are hydrolyzed in the stomach, and all the effects seen on satiety hormone release in such in vivo studies will merely reflect the effect of the hydrolyzed proteins that are present in the duodenum.

Nutrient-induced changes in satiety hormone levels involved in the regulation of satiety are well studied in rats. Keenan et al demonstrated that the use of resistant starch in the diet as a bioactive functional food component is a natural, endogenous way to increase gut hormones that are effective in reducing energy intake (16). Sufian et al showed that pork peptone was able to increase CCK levels in an in vitro assay, and that this peptone also suppressed appetite (17). In our study, duodenal tissue from male rats was used to study the effects of several intact proteins on satiety hormone release. There were no significant changes for both CCK and GLP-1 for all proteins, compared to the negative control. This indicates that the rat intestine is not sensitive for the origin of intact proteins. It may therefore be concluded that the results from prior art studies in the rat may not be extrapolated to humans.

This study demonstrates that intact dietary proteins can directly influence release of the satiety hormones CCK and GLP-1 in human duodenal tissue. We demonstrated that intact pea protein and intact wheat protein elevate both CCK and GLP-1 release in an in vitro study using human duodenal tissue in an Ussing chamber. Both CCK and GLP-1 release is elevated after exposing duodenal tissue to intact pea protein and intact wheat protein. These results are confirmed in another in vitro study using a semi high throughput system for gut hormone secretion. This satiating effect was not observed when pea protein hydrolysates or wheat protein hydrolysates were used.

This lead us to conclude that intact pea protein and intact wheat protein would be suitable for reducing appetite or induce or increase satiety when brought into contact with their receptors in the duodenum. Since it is known that intact proteins hydrolyse in the first part of the gastrointestinal tract, in particular the stomach, intact pea protein and intact wheat protein will not or only very partially exhibit their satiating effect when ingested in a conventional oral preparation. Therefore, special care should be taken to deliver the intact proteins to the duodenum in order for them to arrive there intact.

Substances, in particular proteins, can be delivered intact to the human duodenum in several ways. The skilled person is aware of ways to achieve this goal. As an example, he may use delivery vehicles like capsules, tablets or particles such as micropellets or microparticles.

The invention therefore relates to a composition comprising intact pea protein or intact wheat protein incorporated in a delivery vehicle that increases the resistance of said intact proteins against hydrolysis.

The term incorporated as used herein may be interpreted as meaning encapsulated, included, encompassed or contained.

A delivery vehicle is to be interpreted as a vehicle that is suitable for enteric delivery, i.e. it should be suitable to be swallowed by the target organism, i.e a human or an animal and it should be capable of passing the gastrointestinal tract of the target without getting blocked. Such a vehicle is often referred to as a gastrointestinal delivery vehicle. In all cases, the intact protein composition in the vehicle need to overcome the acidic environment of the stomach. One particularly advantageous way to achieve that goal is to provide the protein with an enteric coating.

The invention thus relates to a composition as described above wherein the delivery vehicle comprises an enteric coating.

Coatings for drug targeting such as enteric coatings do exist in several forms like PH-triggered coatings, pressure-sensitive coatings or time-released coatings [24-29]. For duodenal delivery of relatively large amounts of protein, a pH-sensitive coating used on particles such as micropellets or microparticles is very suitable. Tablets or capsules are also feasible. Particles are preferred however because they are easier to mix with foodstuff and large amounts of protein may be administered in the form of particles whereas the swallowing of large amounts of capsules is often considered problematic and troublesome. Moreover, the contact area of particles may be more advantageous resulting in a slower release of the protein. The use of intact protein compositions encapsulated into particles, such as micropellets or microparticles is thus preferred.

The invention therefore provides a composition as described above wherein the delivery vehicle is a particle. In another embodiment, the invention provides a composition as described above wherein the particles are micropellets or microparticles.

In one embodiment of the present invention, an orally administrable particle containing an intact protein is formed by encapsulating the protein with an enteric coating.

As used herein the term “enteric coating”, is used to mean a material such as a polymer material or materials which encases the core consisting of the active component, in this case the intact pea protein or the intact wheat protein. As such, the polymeric enteric coating material in the present invention does usually not contain any active compound, i.e. intact pea protein or intact wheat protein. Preferably, a substantial amount or the entire enteric polymer coating material is dissolved before the medicament or therapeutically active agent is released from the dosage form, so as to achieve delayed dissolution of the medicament core. A suitable pH-sensitive polymer is one which will dissolve with intestinal juices at the higher pH levels (such as pH greater than 4.5), such as found within the small intestine and therefore permit release of the pharmacologically active substance in the regions of the small intestine and not in the upper portion of the GI tract, such as the stomach.

The polymer coating material is selected such that the therapeutically active agent will be released when the dosage form reaches the small intestine or a region in which the pH is higher, such as more than pH 4.5. Preferred coatings are pH-sensitive materials, which remain intact in the lower pH environs of the stomach, but which disintegrate or dissolve at the pH commonly found in the small intestine of the patient. A very suitable enteric polymer coating material begins to dissolve in an aqueous solution at pH between about 4.5 to about 5.5. The pH-solubility behavior of the enteric polymers as useful in the present invention are usually such that significant dissolution of the enteric polymer coating will not occur until the dosage form has emptied from the stomach. The pH of the small intestine gradually increases from about 4.5 to about 6.5 in the duodenal bulb to about 7.2 in the distal portions of the small intestine (ileum).

In order to provide predictable dissolution corresponding to the small intestine transit time of about 3 hours and permit reproducible release therein, the coating may begin to dissolve within the pH range of the duodenum and continue to dissolve at the pH range within the small intestine. Therefore, the amount of enteric polymer coating may be such that it is substantially dissolved during the approximate three hour transit time within the small intestine.

There are means available in the art to form particles such as micropellets or microparticles from protein preparations. An efficient way to produce such particles has been described in U.S. Pat. No. 6,224,910. The proteins may accordingly be dispersed in an aqueous solution. The aqueous solution may also be sprayed onto nonpareils. Nonpareils are small, usually round particles of pharmaceutically inert materials. Generally, nonpareils that are formed from the combination of sucrose and starch are preferred. One such brand is Nupareils which is sold by Ingredient Technology Corporation. The preferred size is 30-35 mesh although sizes between 4 and 400 mesh may be equally suited, depending on the specific intended use of the eventual particles, micropellets or microparticles.

Alternatively, particles such as micropellets, microparticles or microspheres may also be formed by any other conventional means, even without the addition of filler substances. This allows for the formation of beads with a high load of intact protein. The intact pea protein should be capable of becoming tacky upon moistening or otherwise it should be mixed with minute amounts of suitable binders and optional disintegrants.

Hence, the core of the composition of the invention may also include one or more disintegrants or swelling agents in any practical amount. Conventionally, amounts within the range from about 1% to about 4% by weight of the composition are preferred. Preferred disintegrants or swelling agents are sodium starch glycolate marketed under the trademark EXPLOTAB (Edward Mendell Co.), Ac-Di-Sol (cross-linked sodium carboxymethylcellulose) (FMC Corp), croscarmellose sodium, corn starch, or cross linked polyvinylpyrrolidone.

A major portion of the protein blend may be wet massed extruded and spheronized as is conventionally performed in the art of bead formation whereas a minor portion of the blend may be used for dusting to prevent adhesion and sticking of the beads.

One or more binders may be present in the core in any practical amounts. Conventionally, amounts within the range of from about 0 to about 10% are preferred, even more preferred are amounts of about 1% by weight of the composition. Sodium carboxymethylcellulose is a preferred binder most suitable for use herein. Examples of other binders which may be used include Avicel™ PH101, Avicel™ RC 591, Avicel™ CL-61 1, (FMC Corp), Methocel™ E-5 (Dow Corp.), Starch 1500 (Colorcon, Ltd.), Hydroxypropyl Methylcellulose (HPMC) (Shin-Etsu Chemical Co., Ltd.), Polyvinylpyrrolidone, Potassium Alginate and Sodium Alginate.

Another component which can be added to the intact protein is a stabilizing agent. Stabilizing agents provide physical protection for the protein. Generally these stabilizing agents are inactive water soluble sugars such as lactose, mannitol and trehalose. These act to protect the intact protein during the coating process. One advantageous way to form orally administrable particles such as micropellets or microparticles or microcapsules for use in the present invention is the following. An aqueous solution of the intact protein and the optional stabilizing agent is formed. The aqueous solution may include generally from about 0.5 to about 20% by weight of the intact protein with about 4-8% being preferred, and from about 1% to about 10% by weight of the stabilizing agent with about 5% being preferred.

If the protein solution is to be sprayed on a nonpareil and has a low viscosity, it may be desirable to add 1-10% of polyvinylpyrrolidone to bind the intact protein to the nonpareil.

The nonpareils may be coated with an amount of the aqueous intact protein solution to provide a coating such as for instance of 1-10% protein by weight on a solids basis. Glatt brand powder coater granulators such as the GPCG-1, GPCG-5, or GPCG-60 fluid bed coaters are suitable for use in this application. Coating conditions and times will vary depending on the apparatus and coating viscosity. But, generally coating steps are best conducted at less than 50 degrees Celsius and preferably less than 37 degrees Celsius to avoid denaturing the protein.

Subsequently the particles are coated with a water emulsion of a polymer which upon solidification is acid resistant. This protects the intact protein as it passes through the stomach and releases it into the small intestines where it can act to induce satiety.

The particles or protein coated nonpareils are dried and subsequently coated with an acid stable polymer (enteric coating). Generally, the coating will be applied in the same manner as the protein with the same equipment. The coating composition used in the present invention is preferably a water based emulsion polymer. The preferred coating is an ethylacrylate methacrylic acid copolymer sold under the trademark Eudragit L 30D manufactured by Rhom Pharma. This has a molecular weight of about 250,000 and is generally applied as a 30% aqueous solution. An alternate coating is hydroxypropylmethyl cellulose acetate succinate.

Although Eudragit is the preferred coating polymer, the invention is not limited in this respect and other enteric coating polymers known in the art, such as hydroxypropyl methylcellulose phthalate HP50 (HPMCP-HP50) (USP/NF 220824), HP55 (HPMCP-HP55) (USP/NF type 200731) and HP55S available from Shin Etsu Chemical, Coateric™ (polyvinyl acetate phthalate) (Colorcon Ltd.), Sureteric™ (polyvinyl acetate phthalate) (Colorcon, Ltd.), or Aquateric™ (cellulose acetate phthalate) (FMC Corp.) and the like may be employed.

The coating composition can be combined with a plasticizer to improve the continuity of the coating. There are several well known plasticizers typically used such as triethyl citrate (Citroflex-2), and diethyl phthalate, however, the invention is not limited in this respect and other plasticizers may be used such as triacetin, tributyl sebecate, or polyethylene glycol. Optionally an anti-adherent (anti-agglomerant) which is advantageously a hydrophobic material such as talc, magnesium stearate or fumed silica, with talc being referred, can be applied after coating the beadlet or pellet.

Triethylcitrate (TEC) sold by Morfley Inc. is most preferred. This can form about 1-30% of coating composition. Although plasticizers can be liquid, they are not considered to be solvents since they lodge within the coating altering its physical characteristics. They do not act to dissolve the protein. Any plasticizer which dissolves or denatures the protein would be less suitable. Talc (such as at 3.0% of coating composition) can also be added to prevent sticking between the particles if desired. Also, an antifoaming agent (such as for instance 0.0025% of coating composition) such as sorbitan sesquioleate (Nikko Chemicals Company Limited) or silicone may be added. Both the talc and antifoaming agent may be added if and as much as needed.

The particles comprising the intact protein and optional stabilizing agents, are dried and are then coated with the enteric coating as previously described. The coating solution may be about 30% polymer, 0-30% plasticizer, 0 to 3% talc and 0 to 0.0025% antifoaming agent and water. It is desirable that there are no organic solvents including alcohols and even glycols present in the coating composition. The presence of these solvents during coating application can denature the intact protein. The coating is conducted in the same equipment used to coat the nonpareils with intact protein. The temperature for this coating should be at an optimum to ensure proper coating and as little as possible denaturation of the intact protein. About 30 degrees Celsius but less than 50 degrees Celsius is preferred.

The enteric coated particles then may be administered to a subject in need of treatment according to the invention in any conventional food or feedstuff. It may be mixed with drinks, such as fruit or dairy drinks, such as yoghurt, milk, buttermilk, cream, pudding, but it may also be incorporated in more solid food such as bread, cake, pastry, cheese, chocolate, butter, candy sweets, muesli or candy bars.

Particles, in particular the micropellets or microparticles thus prepared may also be placed in gel capsules for oral administration to humans or animals in need of a treatment for inducing satiety. Dosage will depend on the individual and the course of the therapy.

Particles comprising intact proteins according to the invention may have any size distribution. Usually the size distribution is determined by the intended use. Preferred is a minimum size of 0.01 mm or more such as 0.02, 0.03, 0.04, 0.05, 0.06, 0.07 0.08, 0.09, or 0.1 mm whereas the maximum diameter is determined by the ability of the subject to be treated to swallow the particles. A maximum diameter of 5 mm is preferred; however, less than 4, 3, 2, such as less than 1 millimeter is more preferred.

Microparticles usually range in size between 1 and 100 micrometer, micropellets consist of agglomerates of particles or microparticles and can have any size that is practically useful.

The method according to the invention is most effective when a minimal dose of 0.2 g/kg bodyweight is ingested per day. This is to be interpreted as a dose of 0.2 gram of intact pea protein or intact wheat protein per kg bodyweight of the person ingesting the compound. There is hardly an upper limit, but for practical reasons it is not advisable to ingest more than 10 grams per kg bodyweight per day. Usually the doses range between 0.5 and 5 g/kg bodyweight per day preferably between 0.8 and 2 g/kg bodyweight per day, such as 0.9, 1.0 1.2, 1.4, 1.6, 1.8 g/kg bodyweight per day.

The minimal daily-advised dose for protein intake for non-athletes is 0.8 g/kg bodyweight, and the maximal daily-advised dose of intact pea protein or intact wheat protein for strong-athletes is 2 g/kg bodyweight.

The protein supplement should preferably be taken prior to each meal.

The term intact protein in this context is to be interpreted as non-hydrolysed protein. This means that the protein bonds in the intact protein fraction should be intact, i.e. a degree of hydrolysis (DH) of 0%. The Degree of Hydrolysis (DH) may be determined using a rapid OPA test (Nielsen, P. M.; Petersen, D.; Dambmann, C. Improved method for determining food protein degree of hydrolysis. Journal of Food Science 2001, 66, 642-646).

In order to be effective, a preparation as used in the method according to the invention should contain at least 1% intact protein, preferably more than 10%, more preferably over 20%, 30%, 40% or 50%, even more preferably over 60%, 70%, 80% or 90%, such as 92%, 94%, 96%, 97%, 98% or 99%. Most preferably, the composition comprises 100% intact protein.

In the context of the present invention, the term intact protein is therefore to be interpreted as to mean a preparation comprising at least 1% non-hydrolysed protein, preferably more than 10%, more preferably over 20%, 30%, 40% or 50%, even more preferably over 60%, 70%, 80% or 90%, such as more than 92%, 94%, 96%, 97%, 98% or 99%.

The invention therefore relates to a composition as described above wherein the delivery vehicle comprises between 1% and 100% intact protein as a fraction of the total protein content of the particles.

Intact pea protein or intact wheat protein may be obtained from commercial sources or freshly isolated from wheat or peas. The skilled person is aware of procedures how to obtain intact pea protein or intact wheat protein.

The incorporation of sensitive proteins into particles in order to protect the proteins from hydrolysis is known in the art. With the term “delivery vehicles that protect the proteins against hydrolysis” or “delivery vehicles that increase the resistance of intact proteins against hydrolysis” it is meant that the vehicles such as particles are capable of increasing the resistance of the proteins against hydrolysis such as enzymatic hydrolysis, e.g. by trypsin, chymotrypsin or pepsin or by acid hydrolysis under conditions comparable to a human stomach. Artificially, in a laboratory environment, a suitable test for determining the resistance of proteins against hydrolysis would be the incubation of the proteins at a pH of approximately 1.5 as can be achieved by using more than 0.5N HCL, such as 1N, 2N, or 4N for 10 minutes or more, such as 20 minutes, 30 minutes or 1 hour and then determining the degree of hydrolysis according to the method mentioned above.

Increasing the resistance against hydrolysis in this context means an increase in the fraction of intact proteins versus the fraction of hydrolysed proteins when the proteins are exposed to hydrolyzing conditions as outlined above. Such an increase should be measurable by determining the DH according to methods as describe above. Preferably, the increase should be 10% or more, such as 20% 40%, 60%, 80 or more than 90%. An increase of resistance of 100% would mean that the amount of intact proteins which is protected against hydrolysis is double the amount of intact protein which is not protected against hydrolysis.

In one embodiment, the intact protein according to the present invention is used for the manufacture of a medicament, food supplement, beverage or food product for increasing satiety in a subject.

The invention therefore relates to a composition as described herein for use as a medicament. More in particular, the invention relates to a composition as described herein for use in the treatment and/or prophylaxis of obesity.

As opposed to its use as a medicament, a composition according to the invention may also be used to induce satiety in an otherwise healthy individual, i.e. as a food supplement. The invention therefore relates to a method for inducing or increasing satiety in a human or in an animal, wherein a composition as described herein is administered to said human or animal. Such may be in the form of a food composition comprising a composition as described herein.

As GLP-I slows gastric emptying and inhibits food intake, a longer circulation half-life of GLP-I as a result of enhanced secretion of GLP-I or inhibition of the degradation enzyme DPP-IV will increase satiety in a subject, such that said subject will feel less hungry and have a reduced food intake. In particular, subjects being overweight, such as e.g. obese subjects or subjects being only slightly overweight, will benefit from increased secretion of GLP-I by administration of the intact proteins according to the invention. The medicament, food supplement, beverage or food product can however also be employed to retain a certain weight so as to not get overweight, and may therefore be used to stabilise and/or improve the body weight for cosmetic purposes, i.e. for stabilising and/or improving appearance.

Therefore, in a further embodiment, the intact protein according to the invention is used for the manufacture of a medicament, food supplement, beverage or food product for prophylaxis and/or treatment of obesity.

In another embodiment, the intact protein according to the invention is used for the manufacture of a medicament, food supplement, beverage or food product for lowering of blood glucose levels. It has been found that blood glucose levels are reduced by ingestion of the intact proteins, resulting in improved glucose management, which is particularly advantageous in diabetic subjects.

In a further embodiment, the intact protein according to the invention is used for the manufacture of a medicament, food supplement, beverage or food product for increasing the pancreatic [beta]-cell mass. It has been found that pancreatic [beta]-cell mass increases by ingestion of the intact protein according to the invention results in an improved insulin response and hence an improved glucose management, which is particularly advantageous in diabetic subjects.

In yet a further embodiment, the intact protein according to the invention is used for the manufacture of a medicament, food supplement, beverage or food product for prophylaxis and/or treatment of type 2 diabetes mellitus. Type 2 diabetes mellitus is characterised by resistance to insulin, such that the body does not respond to insulin appropriately, resulting in hyperglycaemia. It is often accompanied by obesity. As GLP-I contributes to normalisation of blood glucose levels as well as to the control of satiety and obesity (body weight), increase of GLP-I levels by increasing the circulation half-life thereof by administration of one or more intact proteins according to the invention will contribute to the prophylaxis and treatment of type 2 diabetes mellitus, and/or will result in improved insulin sensitivity.

For use in a medicament or food supplement, the preparation can be combined with any suitable carrier, diluent, adjuvant, excipient etc., in order to obtain the medicament in the desired administration form. Advantageously, the medicament or food supplement is administered orally. The term “food supplement” is known in the art as any food component which provided specific nutritional or medicinal components and does not provide the full energy value required (i.e. generally less than 2000 or 2500 kcal/day) and includes food supplements in the form of a powder or medicament, as well as health products, such as health drinks. An ingredient that can be added to food before consumption or a preparation that can be consumed as such is also encompassed.

For the intended use, the intact protein according to the present invention may be administered alone or in admixture with a pharmaceutically acceptable carrier, in suitable pharmaceutical formulations which are a further object of the invention. Examples of said formulations, which may be prepared using well known methods and excipients, such as those described in “Remington's Pharmaceutical Sciences Handbook”, Mack Pub. Co., N.Y. U.S.A., are tablets, capsules, syrups, and the like for oral administration, whereas for the parental administration suitable forms are sterile solutions or suspensions in acceptable liquids, implants, etc. The exact dosages will depend on several factors such as type and seriousness of the pathological conditions to be treated, patient's weight and sex, etc. and will be easily determined by the skilled practitioner.

For use in a beverage or food product, the intact proteins according to the present invention can be combined with any common food ingredient. The term “beverage” is meant to include cordials and syrups, as well as formulations of a dry powder to be dissolved in water or another liquid component for the preparation of instant drinks such as juices, soups yoghurt and other dairy stuff.

The present invention is also directed to a method for prophylaxis and/or treatment of any GLP-I mediated condition as discussed above, said method comprising administering an effective amount of the intact protein according to the present invention to a subject in need thereof.

### LEGENDS TO THE FIGURES

FIG. 1: Addition of dietary proteins DPP IV results in decreased activity of the enzyme. Ovomucoid, intact pea protein, egg protein, egg protein hydrolysate, and whey protein inhibited DPP IV activity, with a remaining activity of 83.5%±1.5, 9.6%±0.5, and 67.0%±4.8 respectively. Results are presented as mean±SEM.

FIG. 2: The levels GLP-1 were measured in the supernatant after an exposure time of 2 h. Results are expressed as a percentage of the negative control value and represent mean±SEM of 4 individual experiments. Addition of codfish protein, egg protein, egg protein hydrolysate, sodium casein, intact wheat protein, and whey protein resulted in an increase of GLP-1 release.

FIG. 3: Addition of DPP IV to the negative control resulted in a decreased release of GLP-1. Egg protein hydrolysate, ovomucoid, and intact pea protein in combination with DPP IV inhibit the secretion of GLP-1. All other proteins show no effect on GLP-1 release in combination with DPP IV.

FIG. 4: The levels of CCK were measured in the supernatant of basolateral side of the biopsies in the Ussing Chambers after being exposed to proteins for 2 h to the apical side. Addition of codfish, intact pea protein, or intact wheat protein to human duodenal biopsies increases the release of CCK compared to the negative control. All other proteins doe not show effects on CCK release. Expose of proteins to rat duodenum did not affect CCK release. Results are expressed as mean±SEM.

FIG. 5: The levels of GLP-1 were measured in the supernatant of basolateral side of the biopsies in the Ussing Chambers after being exposed to proteins for 2 h to the apical side. Addition of intact pea protein or intact wheat protein to human duodenal biopsies increases the release of GLP-1 compared to the negative control. All other proteins doe not show effects on GLP-1 release. Expose of proteins to rat duodenum did not affect GLP-1 release. Results are expressed as mean±SEM.

### EXAMPLES

### Example 1

### Materials

DPP IV (human placenta) and DPP aminopeptidase IV substrate hydrochloride were obtained from MP Biomedicals (Uden, the Netherlands). Ovomucoid (from chicken) and soybean were obtained from Worthington Biochemicals (Huissen, the Netherlands). Egg protein hydrolysate, egg protein, wheat hydrolysate, intact wheat protein, whey protein, intact pea protein, casein hydrolysate, sodium casein, and codfish protein were obtained from DSM Food Specialties (Delft, the Netherlands). The cell line used in the study was the STC-1 cell line. This cell line is derived from an endocrine tumor that developed in the small intestine of a double transgenic mouse expressing the rat insulin promotor linked to the simian virus 40 large T antigen and the polyoma virus small t antigen. STC-1 cells (passage 24) were kindly provided by Dr. Douglas Hanahan (University of California, San Francisco). Other reagents used in this study were purchased from Sigma Aldrich unless indicated differently

### Example 2

### Measurement of DPP IV Activity

The DPP IV activity was measured as described by Bauvois (Bauvois B. A collagen-binding glycoprotein on the surface of mouse fibroblasts is identified as dipeptidyl peptidase IV. Biochem J 1988; 252: 723-731.) with some modifications described by Farriol et al. (Farriol M, Pita A M, Fernandez-Bustos M A, Delgado G. Dipeptidyl-peptidase IV in patients with short bowel syndrome. Clin Nutr 2005; 24: 1099-1104.). Briefly, a solution of dehydrated trisodic citrate (10 mM in saline solution pH 6.0) was used as buffer. The enzymatic substrate was DPP aminopeptidase IV substrate hydrochloride (1.11 mM in distilled water). The enzyme assay was performed in a cuvette containing a final volume of 1 ml: 250 μl buffer, 300 μl sample and 450 μl substrate. The reaction mix was monitored before and after an incubation period of 60 min at 37° C. at a wavelength of 450 nm against a negative control. Enzyme and inhibitor were preincubated for 30 min at 37° C. Remaining enzyme activity was measured by adding substrate to a final concentration of 0.5 mM and the reaction was continued for 1 h. Control mixtures lacking enzyme as negative control or inhibitor as positive control were also tested. Remaining activity is expressed as percentage of the control activity (without inhibitor).

### Example 3

### Cell Culture Conditions

STC-1 cells (passage 25 to 40) were maintained in Dulbecco's Modified Eagles Medium (DMEM; Invitrogen) with 10% fecal bovine serum (FBS; Invitrogen), 2 mM L-glutamine, 100 units/ml penicillin, and 100 μg/ml streptomycin as additional supplements; at 37° C. in 5% CO2/air.

### Example 4

### Secretion Studies

Two sets of secretion assays were performed. The first assays were performed to study the direct effects of dietary proteins on the secretion of several satiety hormones. Briefly, tree days before the experiment, STC-1 cells were seeded in 24-well plates (1.0×105 cells/well). On the day of the experiment, cells were first washed 2 times with PBS, followed by addition of a 1% protein-solution to each well. After an incubation period of 2 hours at 37° C., the supernatant was used to measure secreted GLP-1.

In the second set, the indirect effects of the dietary proteins were tested on secretion of satiety hormones. Briefly, on the day of experiment, cells were incubated with a mix of a 1% protein solution with human trypsin (Athens Research; Georgia, USA) or human DPP IV (Athens Research; Georgia, USA), and incubated at 37° C. for 30 min. The supernatant was tested for secreted GLP-1.

### Example 5

### Measurement of GLP-1

GLP-1 levels were determined using RIA (GLP1T-36HK, Linco Research, Missouri, USA). The detection level of this kit is 3 to 333 pM. The intra-assay variation ranges from 10 to 23% and the inter-assay variation from 22 to 38%. There is no cross-reaction with GLP-2 and glucagon (0.01% and 0.2%, respectively).

### Example 6

### Statistical Analysis

The descriptive and statistical analyses were performed with SPSS, version 11.0. The means of the variables are presented with their standard deviation (mean±SD). Comparison of means of the inhibition studies was done using a one-sample t-test. Comparison of means of the secretion studies was done using the one-way ANOVA. A P-value of less than 0.05 was considered statistically significant.

### Example 7

### Inhibition of DPP IV by Dietary Proteins

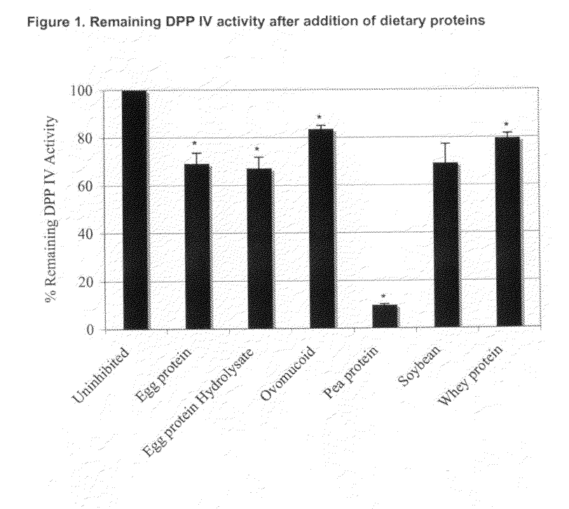

Only six proteins (egg protein, egg protein hydrolysate, ovomucoid, intact pea protein, soybean, and whey protein) were tested for their inhibitory effects on the activity of DPP IV. Ovomucoid, intact pea protein, egg protein, egg protein hydrolysate, and whey protein inhibited DPP IV activity, with a remaining activity of 83.5%±1.5, 9.6%±0.5, and 67.0%±4.8 respectively (FIG. 1).

### Example 8

### Direct Effect of Proteins on GLP-1 Secretion

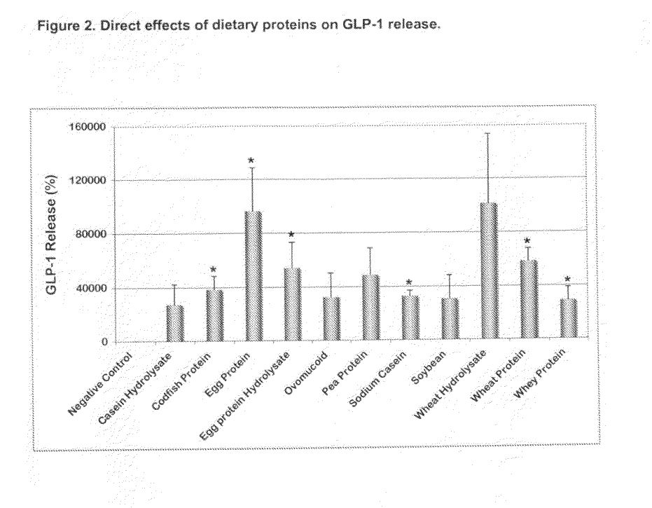

Addition of a 1% protein solution to STC-1 cells resulted in increased levels of GLP-1 in the supernatant compared to the negative control (only HBSS buffer; 100%). Codfish protein, egg protein, egg protein hydrolysate, sodium casein, intact wheat protein, and whey protein were able to significantly elevate the release of GLP-1 into the supernatant (38302%±10181, 96207%±32670, 54120%±19112, 32521%±4524, 58259%±9460, 28879%±9806; respectively) (FIG. 2).

### Example 9

### Effects of DPP IV in Combination with Dietary Proteins in GLP-1 Secretion

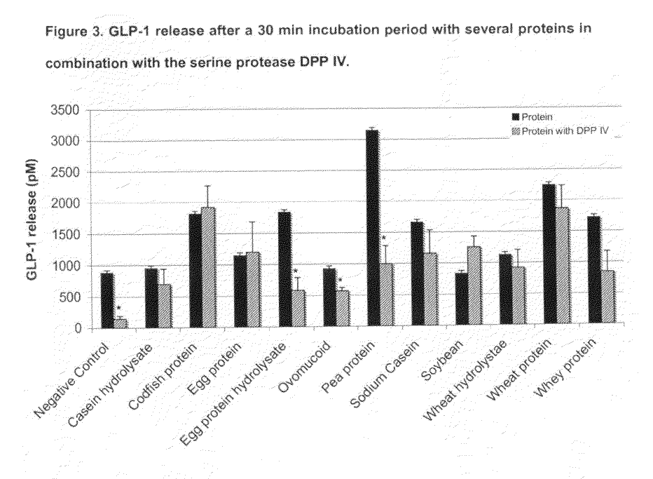

Addition of DPP IV to the negative control resulted in a decreased release of GLP-1 (886 pM to 143 pM±46). Decrease levels of GLP-1 were also observed after combining DPP IV with egg protein hydrolysate (1838 pM to 584 pM±207), ovomucoid (930 pM to 567 pM±56), and intact pea protein (3149 pM to 1002 pM±285). All other proteins show no effect on GLP-1 release in combination with DPP IV (FIG. 3).

### Example 10

### Human Duodenal Biopsy Specimens and Ethics

Eight healthy lean men (mean age 35±16 years), and an average body mass index (BMI) of 23.8±3 kg/m2) were recruited for this study. All were without gastrointestinal disease symptoms. Other exclusion criteria were smoking, ingestion of medicine, or extended alcohol consumption. The Medical Ethical Committee of the University Hospital Maastricht approved the study. All subjects gave their written informed consent prior to participation.

All subjects received a standard evening meal (9 g protein, 39.5 g carbohydrates, 16 g fat per meal) for two days prior to the test day to standardize macronutrient intake. After an overnight fast, mucosal tissue samples from the horizontal part of the duodenum were obtained by flexible gastroduodenoscopy using standard biopsy forceps. During this procedure, no sedatives were given to the subjects.

### Example 11

### Preparation of Animal Duodenal Tissue

Ten male Lewis rats, at least 100 days of age, were used in this study. The rats had free access to food and water prior to sacrifice. After sacrifice, the duodenum was placed in ice-cold Krebs-Ringer bicarbonate buffer (KRB) and arrived at the laboratory within 15 min. The muscle layer was removed, and mucosal tissue samples were obtained with a standard biopsy forceps, equal to the forceps used in the human experiments.

### Example 12

### Ussing Chamber Experimental Procedure

The tissue samples were mounted in modified 1.5 ml Ussing Chambers (Harvard Apparatus Inc., Holliston, Mass., USA) with a Ø 9-mm opening and reduced to an exposed tissue area of 1.76 mm2, using a technique previously described by Wallon et al (10). The tissue segments were mounted between two 0.4 mm polyester films with a Ø 1.5 mm opening with round edges. The flexibility of the films reduced squeezing of the tissues at the border of the openings to minimize edge damage. The surface of the polyester was roughened with a fine abrasive paper to keep the tissue segments in position. After mounting, each half chamber was filled with 1.5 ml KRB, bathing both the mucosal and serosal side of the specimen. The KRB solution was continuously oxygenated with O2/CO2 (95%/5%) and stirred by gas flow in the chambers. pH was kept at 7.4 at a temperature of 37° C. with a heater block system. After a 40 min equilibration period to achieve steady state conditions regarding potential difference (PD), the KRB in the mucosal compartment was replaced with KRB containing 0.1 mg/ml protein (codfish, egg, ovomucoid, pea, or intact wheat protein), and that in the serosal compartment was replaced with fresh KRB. PD, transmucosal electrical resistance (TER) and short-circuit current (Isc) were followed over a 120 min period. Experiments were done in open-circuit conditions with assessment of electrophysical parameters at 1 min intervals. In this experiment, samples of the apical side of the tissue segments were taken at the end of the experiment (after 2 h) for CCK- and GLP-1 analysis.

### Example 13

### Electrical Measurements

A four-electrode system was used, as described previously. One pair of Ag/CI electrodes with 3M NaCl/2% agar bridges was used for measurement of transepithelial PD and another pair of Ag/Cl electrodes was used to monitor current. The electrodes were coupled to an external 6-channel electronic unit with a voltage controlled current source. Data sampling was computer controlled via an A/D D/A board (iworx IX/118, Iworx, Dover, USA), using the Labscribe program. Every other minute, direct pulses of −3.3, and 0 μA with a duration of 2 seconds each were sent across the tissue segments and the voltage response was measured. In each measurement, the mean voltage response of 2 seconds was calculated. A linear-squares fit was performed on the current (I)-voltage (U) pair relationship: U=PD+TER×I. The TER was obtained from the slope of the line and the PD from the intersection of the voltage.

### Example 14

### Measurement of CCK and GLP-1

CCK and GLP-1 levels were determined using radio immuno assays (RIA). Briefly, a fixed concentration of unlabeled antigen is incubated with a constant dilution of antiserum such that the concentration of antigen binding sites on the antibody is limited. Addition of labeled tracer to this system results in a competition between labeled tracer and unlabeled antigen for the limited and constant number of binding sites on the antibody. Thus, the amount of tracer bound to antibody will decrease as the concentration of unlabeled antigen increases. This can be measured after separating antibody-bound from free tracer and counting the pellet. CCK levels were determined using the RIA from Euria-CCK, Euro-Diagnostica AB, Malmö, Sweden. According to the manufacturers instructions, the detection level of this kit was 0.78 pmol/L. The intra-assay variation ranges from 2.0 to 5.5% and the inter-assay variation from 4.1 to 13.7%. Cross-reaction with gastrin is ≦0.5%. GLP-1 levels were determined using the RIA from Linco Research, Missouri, USA. The detection level of this kit was 3 to 333 pM. The intra-assay variation ranges from 10 to 23% and the inter-assay variation from 22 to 38%. There is no cross-reaction with GLP-2 and glucagon (0.01% and 0.2%, respectively).

### Example 15

### Statistical Analyses

The descriptive and statistical analyses were performed with SPSS, version 11.0. The means of the variables are presented with their standard error (mean±SEM). Comparison of the electrophysiological parameters was done using the Wilcoxon signed rank test. Comparison of means of the secreted hormones was done using an unpaired Student t-test. A P-value of less than 0.05 was considered statistically significant.

### Example 16

### Hormone Release

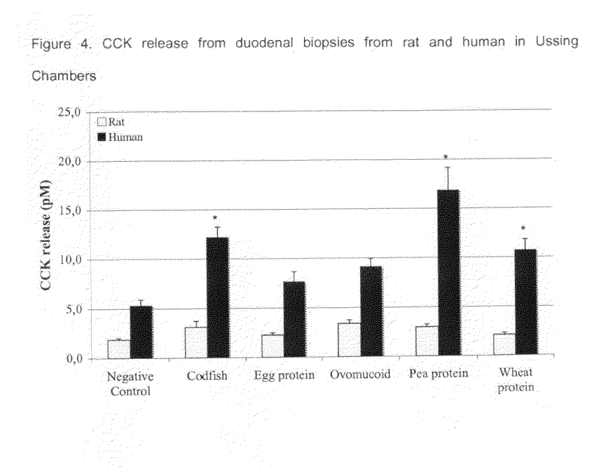

We observed basal secretion of CCK without any stimulation of proteins (rat: 1.9 pM±0.2; human: 5.3 pM±0.6) (FIG. 4). Addition of the different proteins to the apical side of rat duodenum did not influence release of CCK by rat duodenum, but addition of codfish, intact pea protein, or intact wheat protein to human duodenal tissue induced elevated levels of CCK in the supernatant (12.2 pM±1; 16.9 pM±2.3; 10.7 pM±1; respectively). Egg protein and ovomucoid did not affect CCK release in human duodenum.

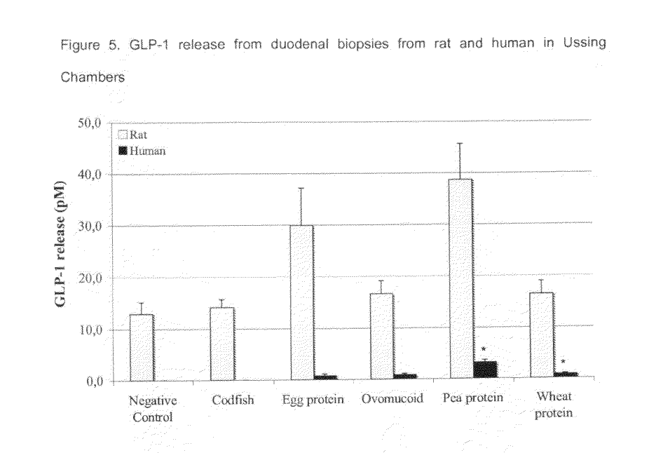

FIG. 5 shows the release of GLP-1 from duodenal tissue into the supernatant. Rat duodenal mucosa was able to secrete GLP-1 without stimulation by protein (113 pM±2.2), whereas human mucosa was not able to secrete GLP-1 under basal conditions. Addition of proteins did not affect the release of GLP-1 in rat biopsies. However, addition of intact pea protein or wheat increased the GLP-1 release into the supernatant (3.2 pM±0.5; 0.9 pM±0.2; respectively) compared to the negative control in human biopsies.

### Example 17

### Electrical Measurements

The electrical parameters PD, Isc, and TER were followed over time. Table 1 represents the basal electrical properties of all biopsies taken. After an equilibration period of 40 min, the mean PD of rat tissue was −0.9 mV±0.03; the TER 17.3 Ω·cm2±0.8; and the Isc was 54.9 μA/cm2±2.9. In human tissue we observed a PD of −1.6 mV±0.2; the mean TER was 38 Ω·cm2±1.6; and the mean Isc was 44 μA/cm2±5.1.

Directly after addition of protein to the apical side, an increase in TER and a decrease in Isc were observed in both rat and human tissue for all proteins. After 60 min and 120 min the TER decreased, whereas the Isc increased for all proteins. There were no significant changes over time, compared to the negative control.

TABLE 1
Basal electrical properties of rat and human duodenum

| | Rat | Human |
|---|---|---|
| | (n = 60) | (n = 48) |
| PD (mV) | −0.9 ± 0.03 | − 1.6 ± 0.2 |
| TER (Ω · cm²) | 17.3 ± 0.8 | 38 ± 1.6 |
| Isc (μA/cm²) | 54.9 ± 2.9 | 44 ± 5.1 |

Baseline electrophysiological parameters of duodenal biopsies for rat and human.
PD: potential difference;
TER: trans epithelial resistance;
Isc: short circuit current.
Results are expressed as means ± SEM.

Table 2 shows the electrical properties of the rat tissue and Table 3 shows the electrical properties of human tissue over a time period of 120 minutes.

### Example 18

### Intact Pea Protein Coated Nonpareils

Intact pea protein coated nonpareils were prepared from 20 grams of nonpareils, 1 gram of intact pea protein (Seek Natural, St. Albans, Herts) and 1 gram of lactose. These were then coated with Eudragit L30D in a total aqueous system (7 grams Eudragit L30D and 22 grams coated nonpareils). These particles are resistant to acid pH conditions typically encountered in the gastric juices. Intact protein is not released until the pH approached 6. At pH 6 to 7, substantially all of the intact pea protein is released. To determine the release of intact pea protein over time, these particles may be exposed to either intestinal pH of 6.8 or gastric pH of 1.2. At the gastric pH of 1.2, virtually none of the intact pea protein will be released whereas at pH 6.8, substantially all of the intact pea protein will be released in a short time.

### Example 19

### Food Containing Intact Pea Protein

The pea proteins encapsulated in enteric coated particles as prepared in Example 18 may be mixed with yoghurt. An amount of 80 grams of the particle preparation of example 18 may be added to a portion of 200 grams of yoghurt and administered to a human in order to reduce satiety.

TABLE 2
Effects of several dietary proteins on TER and Isc of rat duodenum over time

| | T = 0 min | | t = 60 min | | t = 120 min | |
|---|---|---|---|---|---|---|
| | ΔTER | ΔIsc | ΔTER | ΔIsc | ΔTER | ΔIsc |
| | (Ω · cm²) | (μA/cm²) | (Ω · cm²) | (μA/cm²) | (Ω · cm²) | (μA/cm²) |
| Negative | 2.6 ± 0.7 | −6.0 ± 3.1 | −2.7 ± 0.3 | 12.5 ± 4.3 | −5.1 ± 0.6 | 22.2 ± 6.4 |
| Control | | | | | | |
| Codfish | 2.7 ± 0.5 | −12.2 ± 3.0 | −4.2 ± 0.5 | 8.6 ± 3.9 | −7.3 ± 0.8 | 21.9 ± 6.4 |
| Egg protein | 2.4 ± 0.5 | −10.6 ± 3.1 | −2.8 ± 0.7 | 11.1 ± 3.8 | −4.8 ± 1.1 | 23.0 ± 6.4 |
| Ovomucoid | 1.8 ± 1.0 | −12.2 ± 3.2 | −6.4 ± 3.4 | 13.9 ± 4.0 | −5.4 ± 1.1 | 13.2 ± 7.1 |
| Intact pea | 3.7 ± 2.0 | −11.7 ± 3.3 | −2.7 ± 0.2 | 11.4 ± 4.0 | −4.7 ± 0.4 | 20.9 ± 7.2 |
| protein | | | | | | |
| Intact wheat | 3.2 ± 1.3 | −14.3 ± 2.4 | −3.5 ± 0.4 | 3.6 ± 3.5 | −6.8 ± 1.3 | 8.7 ± 6.8 |
| protein | | | | | | |

The electrophysiological parameters of duodenal biopsies of rat was followed over time after addition of different proteins. At time point 0 min the proteins were added to the apical side of the tissue. No significant electrophysiological changes were observed over time for all proteins compared to the negative control.
Results are expressed as means ± SEM.

TABLE 3
Effects of several dietary proteins on TER and Isc of human duodenum over time

| | t = 0 min | | t = 60 min | | t = 120 min | |
|---|---|---|---|---|---|---|
| | ΔTER | ΔIsc | ΔTER | ΔIsc | ΔTER | ΔIsc |
| | (Ω · cm²) | (μA/cm²) | (Ω · cm²) | (μA/cm²) | (Ω · cm²) | (μA/cm²) |
| Negative | 2.5 ± 2.2 | 5.6 ± 6.7 | −6.6 ± 1.4 | −8.8 ± 7.5 | −9.5 ± 1.7 | −7.2 ± 9.6 |
| Control | | | | | | |
| Codfish | 3.8 ± 1.3 | 0.5 ± 5.4 | −5.5 ± 1.2 | 13.6 ± 7.3 | −9.5 ± 1.7 | 11.3 ± 9.7 |
| Egg protein | 4.1 ± 1.2 | 0.6 ± 5.4 | −10.7 ± 1.3 | 22.0 ± 7.7 | −13.9 ± 2.0 | 20.8 ± 9.8 |
| Ovomucoid | 4.7 ± 1.2 | −4.1 ± 5.7 | −10.4 ± 1.2 | 11.9 ± 7.8 | −14.8 ± 1.9 | 17.6 ± 9.4 |
| Intact pea | 3.8 ± 1.9 | −7.9 ± 5.9 | −6.3 ± 1.6 | 9.6 ± 5.2 | −8.9 ± 2.0 | 11.9 ± 7.5 |
| protein | | | | | | |
| Intact wheat | 5.4 ± 1.9 | −4.8 ± 5.8 | −8.1 ± 1.7 | 3.5 ± 5.0 | −12.4 ± 2.0 | 2.5 ± 6.7 |
| protein | | | | | | |

The electrophysiological parameters of duodenal biopsies of human was followed over time after addition of different proteins. At time point 0 min the proteins were added to the apical side of the tissue. No significant electrophysiological changes were observed over time for all proteins compared to the negative control.
Results are expressed as means ± SEM.

### LITERATURE AND REFERENCES

1. Cummings D E, Overduin J. Gastrointestinal regulation of food intake. J Clin Invest 2007; 117:13-23.
2. Brubaker P L, Anini Y. Direct and indirect mechanisms regulating secretion of glucagon-like peptide-1 and glucagon-like peptide-2. Can J Physiol Pharmacol 2003; 81:1005-12.
3. Saris W H, Tarnopolsky M A. Controlling food intake and energy balance: which macronutrient should we select? Curr Opin Clin Nutr Metab Care 2003; 6:609-13.
4. Tome D. Protein, amino acids and the control of food intake. Br J Nutr 2004; 92 Suppl 1:S27-30.
5. Westerterp-Plantenga M S, Lejeune M P. Protein intake and body-weight regulation. Appetite 2005; 45:187-90.
6. Bensaid A, Tome D, Gietzen D, et al. Protein is more potent than carbohydrate for reducing appetite in rats. Physiol Behav 2002; 75:577-82.
7. Hug M J. Transepithelial measurements using the Ussing chamber. 2002.
8. Boudry G. The Ussing chamber technique to evaluate alternatives to in-feed antibiotics for young pigs. anim. Res. 2005; 54:219-230.
9. Davis J L, Little D, Blikslager A T, Papich M G. Mucosal permeability of water-soluble drugs in the equine jejunum: a preliminary investigation. J Vet Pharmacol Ther 2006; 29:379-85.
10. Wallon C, Braaf Y, Wolving M, Olaison G, Soderholm J D. Endoscopic biopsies in Ussing chambers evaluated for studies of macromolecular permeability in the human colon. Scand J Gastroenterol 2005; 40:586-95.
11. Westerterp-Plantenga M S, Luscombe-Marsh N, Lejeune M P, et al. Dietary protein, metabolism, and body-weight regulation: dose-response effects. Int J Obes (Lond) 2006; 30 Suppl 3:S16-23.
12. van Erk M J, Blom W A, van Ommen B, Hendriks H F. High-protein and high-carbohydrate breakfasts differentially change the transcriptome of human blood cells. Am J Clin Nutr 2006; 84:1233-41.
13. Diepvens K, Haberer D, Westerterp-Plantenga M. Different proteins and biopeptides differently affect satiety and anorexigenic/orexigenic hormones in healthy humans. Int J Obes (Lond) 2008; 32:510-8.
14. Weickert M O, Spranger J, Holst J J, et al. Wheat-fibre-induced changes of postprandial peptide YY and ghrelin responses are not associated with acute alterations of satiety. Br J Nutr 2006; 96:795-8.
15. Borzoei S, Neovius M, Barkeling B, Teixeira-Pinto A, Rossner S. A comparison of effects of fish and beef protein on satiety in normal weight men. Eur J Clin Nutr 2006; 60:897-902.
16. Keenan M J, Zhou J, McCutcheon K L, et al. Effects of resistant starch, a non-digestible fermentable fiber, on reducing body fat. Obesity (Silver Spring) 2006; 14:1523-34.
17. Sufian M K, Hira T, Miyashita K, Nishi T, Asano K, Hara H. Pork peptone stimulates cholecystokinin secretion from enteroendocrine cells and suppresses appetite in rats. Biosci Biotechnol Biochem 2006; 70:1869-74.
18. Lang V, Bellisle F, Oppert J M, et al. Satiating effect of proteins in healthy subjects: a comparison of egg albumin, casein, gelatin, soy protein, intact pea protein, and wheat gluten. Am J Clin Nutr 1998; 67:1197-204.
19. Uhe A M, Collier G R, O'Dea K. A comparison of the effects of beef, chicken and fish protein on satiety and amino acid profiles in lean male subjects. J Nutr 1992; 122:467-72.
20. Lieverse R J, Masclee A A, Jansen J B, Lam W F, Lamers C B. Obese women are less sensitive for the satiety effects of bombesin than lean women. Eur J Clin Nutr 1998; 52:207-12.
21. Larsen R, Mertz-Nielsen A, Hansen M B, Poulsen S S, Bindslev N. Novel modified Ussing chamber for the study of absorption and secretion in human endoscopic biopsies. Acta Physiol Scand 2001; 173:213-22.
22. Grass G M, Sweetana S A. In vitro measurement of gastrointestinal tissue permeability using a new diffusion cell. Pharm Res 1988; 5:372-6.
23. Reims A, Strandvik B, Sjovall H. Epithelial electrical resistance as a measure of permeability changes in pediatric duodenal biopsies. J Pediatr Gastroenterol Nutr 2006; 43:619-23.
24. Bodmeier, R., H. G. Chen, and O. Paeratakul, A novel approach to the oral delivery of microparticles. Pharm Res, 1989. 6(5): p. 413-7.
25. Dhaliwal, S., et al., Mucoadhesive microspheres for gastroretentive delivery of acyclovir: in vitro and in vivo evaluation. Aaps J, 2008. 10(2): p. 322-30.
26. Ichikawa, H. and Y. Fukumori, [Design of nanohydrogel-incorporated microcapsules for appropriate controlled-release of peptide drugs]. Yakugaku Zasshi, 2007. 127(5): p. 813-23.
27. Mustata, G. and S. M. Dinh, Approaches to oral drug delivery for challenging molecules. Crit Rev Ther Drug Carrier Syst, 2006. 23(2): p. 111-35.
28. Clear, N. J., et al., Evaluation of the Intelisite capsule to deliver theophylline and frusemide tablets to the small intestine and colon. Eur J Pharm Sci, 2001. 13(4): p. 375-84.
29. Malik, D. K., et al., Recent advances in protein and peptide drug delivery systems. Curr Drug Deliv, 2007. 4(2): p. 141-51.

1. A composition comprising intact pea protein or intact wheat protein incorporated in a delivery vehicle that increases the resistance of said intact proteins against hydrolysis.

2. A composition according to claim 1 wherein the delivery vehicle comprises an enteric coating.

3. A composition according to claim 1 wherein the delivery vehicle is a particle.

4. A composition according to claim 3 wherein the particles are micropellets or microparticles.

5. A composition according to claim 1 wherein the delivery vehicle comprises between 1% and 100% intact protein as a fraction of the total protein content of the vehicle.

6. A composition according to claim 1 for use as a medicament.

7. A composition according to claim 1 for use in the treatment and/or prophylaxis of obesity.

8. Method for inducing or increasing satiety in a human or in an animal, wherein a composition according to claim 1 is administered to said human or animal.

9. Food composition comprising a composition according to claim 1.
